(19) United States
(12) Patent Application Publication
(10) Pub. No.: US 2008/0103193 A1
(43) Pub. Date: May 1, 2008

(54) Methods for making compositions and compositions for treating pain and cachexia

(76) Inventors: Trevor Percival Castor, Arlington, VA (US); Lorraine C. Rosenberry, Newcastle, ME (US); Theodore A. Tyler, Framingham, MA (US); Richard J. Student, Hubbardston, MA (US)

Correspondence Address: Aphios Corporation, 3-E Gill Street, Woburn, MA, 01801, US

(21) Appl. No.: 11/586,889

(22) Filed: Oct. 26, 2006

(51) Int. Cl. A61K 31/353; C07D 311/80

(52) U.S. Cl. 514/454; 549/390

(57) ABSTRACT

The present invention pertains to methods for making compositions and compositions for treating pain and cachexia or AIDS wasting. In particular, the instant invention employs methods for making Δ9-tetrahydrocannabinol (Δ9-THC) and Δ9-tetrahydrocannabinolic acid (Δ9-THCA) from Cannabis sativa, and compositions for the treatment of these diseases.

### GOVERNMENT SUPPORT

Research leading to this invention was in part funded with Grant No. IR43CA092855-01A1 from the National Cancer Institute, National Institutes of Health, United States of America.

### FIELD OF THE INVENTION

The present invention pertains to methods for making compositions and compositions for treating pain and cachexia or AIDS wasting. In particular, the instant invention employs methods for making Δ9-tetrahydrocannabinol (Δ9-THC) and Δ9-tetrahydrocannabinolic acid (Δ9-THCA) from Cannabis sativa, and compositions for the treatment of these diseases.

### BACKGROUND OF THE INVENTION

On Feb. 19 and 20, 1997, the National Institutes of Health convened an Ad Hoc Expert Panel to discuss current knowledge of the medical uses of Cannabis. The report compiled by the Ad Hoc Group of Experts, stated that they “favored the development of alternative dosage forms, including an inhaler dosage form into which a controlled unit-dose of THC could be placed and volatilized.” The report discussed the importance of other cannabinoids and their potential interaction effects upon THC, stating: “Varying proportions of other cannabinoids, mainly cannabidiol (CBD) and cannabinol (CBN), are also present in Cannabis, sometimes in quantities that might modify the pharmacology of THC or cause effects of their own. CBD is not psychoactive but has significant anticonvulsant, sedative, and other pharmacological activity likely to interact with THC.” The Institute of Medicine (IOM) released its report Mar. 17, 1999 with two conclusions and recommendations that apply to this research:
Conclusion 1: The variety of mechanisms through which cannabinoids can influence human physiology underlies the variety of potential therapeutic uses for drugs that might act selectively on different cannabinoid systems.
Recommendation 1: Research should continue into the physiological effects of synthetic and plant-derived cannabinoids . . . . Because different cannabinoids appear to have different effects, cannabinoid research should include, but not be restricted to, effects attributable to THC alone.
Conclusion 2: Scientific data indicate the potential therapeutic value of cannabinoid drugs, primarily THC, for pain relief, control of nausea and vomiting, and appetite stimulation; smoked Cannabis, however, is a crude THC delivery system that also delivers harmful substances.
Recommendation 2: Clinical trials of cannabinoid drugs for symptom management should be conducted with the goal of developing rapid-onset, reliable, and safe delivery systems.

The development of natural, complex cannabinoid pharmaceuticals is significant because they could provide new tools for studying the physiological effects and the therapeutic value of cannabinoids in humans, potentially leading to new therapeutic agents that could benefit a number of patients. Although the therapeutic values of some cannabinoids are known, only two forms of cannabinoids, legal Dronabinol and illegal Cannabis, have been used to date for medical treatments and both are significantly flawed. Dronabinol suffers from high incidence of adverse side effects and low bioavailability (only 6-20 percent reaches systemic circulation) due to poor solubility of highly lipophilic THC in aqueous solution and high first-pass metabolism in the liver. A metabolite of orally dosed Dronabinol, 11-OH-A-9-THC, created during the digestive processes is three times more psychoactive than THC itself, with no indication of added medical benefit. Onset of action is slow with peak plasma concentrations attained 2-4 hours after dosing. The most common adverse events associated with Dronabinol involve the central nervous system, including anxiety, confusion, depersonalization, dizziness, euphoria, dysphoria, somnolence and thinking abnormality.

When Cannabis is smoked, cannabinoids are rapidly absorbed through the alveoli into the bloodstream, traveling directly to receptors in the brain. However, dangerous tars and other combustion byproducts are deposited in the lungs, making smoking—apart from its illegality—an unacceptable delivery method. In addition, cannabinoid composition in Cannabis is not consistent. The plant contains many complex variables that would make it difficult, if not impossible, to dose. Current knowledge about the medical use of cannabinoids is extrapolated almost entirely from studies involving smoked or ingested raw Cannabis and orally dosed synthetic THC. Groups of patients suffering from pain, nausea, cachexia, spasticity and/or Alzheimer's may benefit from new cannabinoids. “The accumulated data from advances in cannabinoid science of the last 16 years suggest a variety of indications, particularly pain and nausea relief, and appetite stimulation where cannabinoid administrations would prove useful. For patients such as those with AIDS or undergoing chemotherapy who suffer simultaneously from severe pain, nausea, and appetite loss, cannabinoid drugs might offer broad spectrum relief not found in any other single medication.” (IOM Report, 1999).

In 1964 Gaoni and Mechoulam reported the isolation, structure and partial synthesis of the pure form of Δ9-tetrahydrocannabinol, (THC), the most psychoactive cannabinoid. By 1980 Turner, Elsohly and Boeren of the Research Institute of Pharmaceutical Sciences at the School of Pharmacy, University of Mississippi, reported that 425 compounds had been isolated from Cannabis sativa L. including sixty-one cannabinoids. Most of these cannabinoids were isolated by extracting the raw biomass with organic solvents such as hexane, chloroform and petroleum ether followed by chromatographic purification on silica. As described below, these processes are very complex and lengthy resulting in significant loss of product and low yields while utilizing large quantities of toxic organic solvents.

In 1970, Davis et al. reported a large-scale process for preparing cannabis concentrates as well as enriched and pure cannabinoids. Batches of Cannabis was processed in the following way: (1) each batch was extracted four times with 95% ethanol over a 7 day period; (2) each alcohol extract was concentrated in vacuo on precision stills at temperatures less than 40° C.; (3) each resulting concentrate was treated with ½ volume of water and 1 volume of hexane; (4) the lower phase was withdrawn and re-extracted with hexane; (5) the hexane phases were combined and backwashed with 50% aqueous alcohol; (6) the hexane extracts were then separately evaporated in vacuo at temperatures less than 40° C. to a paste; (7) some of the first hexane extract was mixed with cottonseed oil and further processed by molecular distillation, yielding 17.74% Δ9-THC, 1.60% cannabidiol, and 4.72% cannabinol; (8) in an alternate procedure, some of the hexane extract was chromatographed on Florisil using 2% methanol in hexane, to yield approximately 20% Δ9-THC, 2% cannabidiol, and 5% cannabinol; (9) molecular distillation of the cannabinoid fraction gave an oil which contained 35% Δ9-THC and amounted to 55-65% of the total distillate; and (10) the relative ratios of the Δ9-THC, cannabidiol and cannabinol remained essentially unchanged by this process of chromatography and molecular distillation.

A large body of scientific information exists that elucidates extraction methodologies used to derive various cannabinoids from a large number of Cannabis plant variants for identification, characterization and structure determination purposes. The known methodologies and structures are invaluable for future scientific research. These isolation technologies are not, however, appropriate for the manufacturing of pharmaceutical grade Δ9-THC in commercial quantities. In general, the manufacturing of Δ9-THC by conventional organic solvents is a complex and lengthy multi-step process that will reduce overall yields and increase costs while having adverse effects on the environment from volatile organic solvents and potentially toxic organic wastes.

In this invention, natural Δ9-THC cannabinoid products are isolated by utilizing supercritical, critical or near-critical fluids with or without polar cosolvents. As shown in FIG. 1, a compound becomes supercritical at conditions that equal or exceed both its critical temperature and critical pressure. These parameters are intrinsic thermodynamic properties of all sufficiently stable pure compounds and mixtures. Carbon dioxide, for example, becomes a supercritical fluid at conditions that equal or exceed its critical temperature of 31.1° C. and its critical pressure of 72.8 atm (1,070 psig). In the supercritical fluid region, normally gaseous substances such as carbon dioxide become dense phase fluids that have been observed to exhibit greatly enhanced solvating power.

At a pressure of 3,000 psig (204 atm) and a temperature of 40° C., carbon dioxide has a density of approximately 0.8 g/cc and behaves much like a nonpolar organic solvent such as hexane, having a dipole moment of zero debyes. A supercritical fluid uniquely displays a wide spectrum of solvation power, as its density is strongly dependent upon temperature and pressure. Temperature changes of tens of degrees or pressure changes by tens of atmospheres can change a compound's solubility in a supercritical fluid by an order of magnitude or more. This unique feature allows for the fine-tuning of solvation power and the fractionation of mixed solutes. The selectivity of nonpolar supercritical fluid solvents can also be enhanced by the addition of compounds known as modifiers (also referred to as entrainers or cosolvents). These modifiers are typically somewhat polar organic solvents such as acetone, ethanol and methanol.

In addition to their unique solubilization characteristics, supercritical fluids possess other physicochemical properties that add to their attractiveness as solvents. They can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields. While similar in many ways to conventional nonpolar solvents such as hexane, it is well known that these fluids can extract a different spectrum of materials than conventional techniques. Product volatilization and oxidation as well as processing time and organic solvent usage can be significantly reduced with the use of supercritical fluids.

A material at conditions that border its supercritical state will have properties that are similar to those of the substance in the supercritical state. These so-called “near critical” fluids are also useful. To simplify the terminology, materials that are utilized under conditions, which are supercritical, near critical, or exactly at their critical point with or without polar cosolvents or entrainers are jointly referred to as SuperFluids™ or “SFS.”

SuperFluids™ can also be utilized for the isolation and purification of the carboxylic acid precursor to Δ9-THC, Δ9-THCA. The latter is the major component of untreated (not heated to 100° C. or greater) Cannabis sativa biomass. Δ⁹-THCA is stoichometrically converted to Δ9-THC when heated to more than 100° C., and is thus an ideal candidate for an enhanced drug delivery device to deliver Δ9-THC to the lung, avoiding the tars and other combustion byproducts generated from smoking marijuana or the low bioavailability (6-20%), high first pass metabolism in the liver and the generation of highly psychoactive metabolites associated with the oral intake of synthetic Δ9-THC capsules.

SuperFluids™ can also be utilized for the isolation and purification of other natural cannabinoids such as cannabidiol and cannabinol.

### SUMMARY

The present invention pertains to methods for making compositions and compositions for treating pain and cachexia or A/DS wasting. In one aspect of the present invention, the compositions comprise of Δ9-tetrahydrocannabinol (Δ9-THC) and Δ9-tetrahydrocannabinolic acid (Δ9-THCA). We discovered that SFS can be utilized to purify Δ9-THC and Δ9-THCA in high absolute purities and high overall yields in very short times (<4 hours). We demonstrated that this single-step extraction and chromatographic purification could be readily coupled to proprietary segmentation chromatography systems for the manufacture of 99+% pure Δ9-THC and Δ9-THCA in a cost-effective and relatively environmentally benign manner. The developed processes are innovative because there are no commercial processes of the isolation of natural Δ9-THC, and no commercial process for either the synthetic manufacture or natural isolation of Δ9-THCA. The developed process will be capable of isolating and manufacturing other natural cannabinoids such as pure (99+%) cannabidiol and cannabinol for research as well as potential clinical use. Additionally, certain aspects of the SFS process and the segmentation chromatography systems are unique to the isolation of cannabinoids from Cannabis. The use of Δ9-THCA for the inhaled delivery of Δ9-THC to the lung, significantly improving bioavailability as well as avoiding high first pass metabolism in the liver and the generation of highly psychoactive metabolites, is also unique.

### BRIEF DESCRIPTION OF THE DRAWINGS

FIG. 1 is a depiction of a supercritical fluid phase diagram.

FIG. 2 is a process flow diagram of the SFS extraction apparatus.

FIG. 3 is a process flow diagram of the SFS chromatography apparatus.

FIG. 4 is regression curves of cannabinoid standards.

FIG. 5 is an HPLC chromatogram of SFS extraction of untreated Cannabis [MAJ-8-2].

FIG. 6 is an HPLC chromatogram of SFS extraction of heat-treated Cannabis [MAJ-21-3S].

FIG. 7 is a process flow diagram of bench top SFS extraction and purification (CXP) apparatus.

FIG. 8 is a bar chart of the SFS-CXP of heat treated Cannabis [MAJB-4]

FIG. 9 is an HPLC chromatogram of SFS-CXP of heat-treated Cannabis [MAJB-4-F2].

FIG. 10 is an HPLC chromatogram of SFS-CXP of untreated Cannabis [MAJB-3-F8].

FIG. 11 is a five-column segmentation chromatography system

FIG. 12 is a graph of log (k) versus methanol for cannabinoids on C18.

FIG. 13 is a bar chart of a segmentation chromatographic separation of Δ9-THC, Δ9-THCA and CBN on CG-71.

FIG. 14 is an HPLC chromatogram of Δ9-THC [TT-38-C2-F4].

FIG. 15 is an HPLC chromatogram of Δ9-THCA [TT-10-21-03].

### DETAILED DESCRIPTION

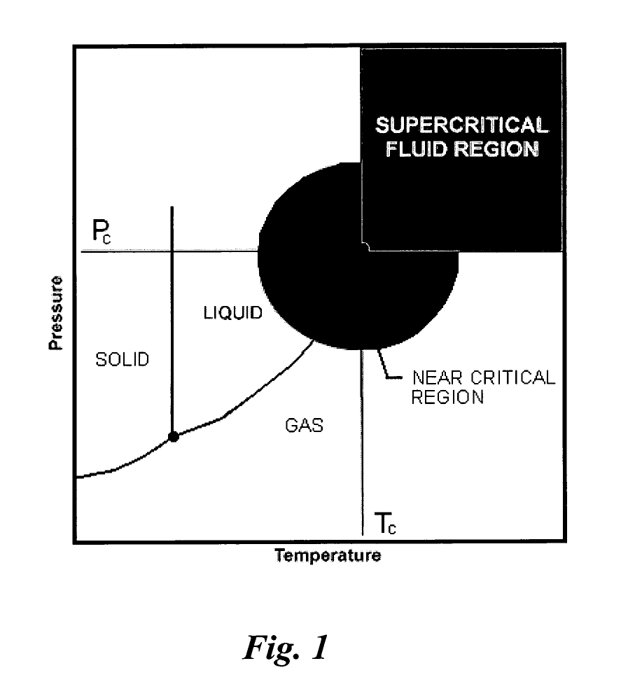

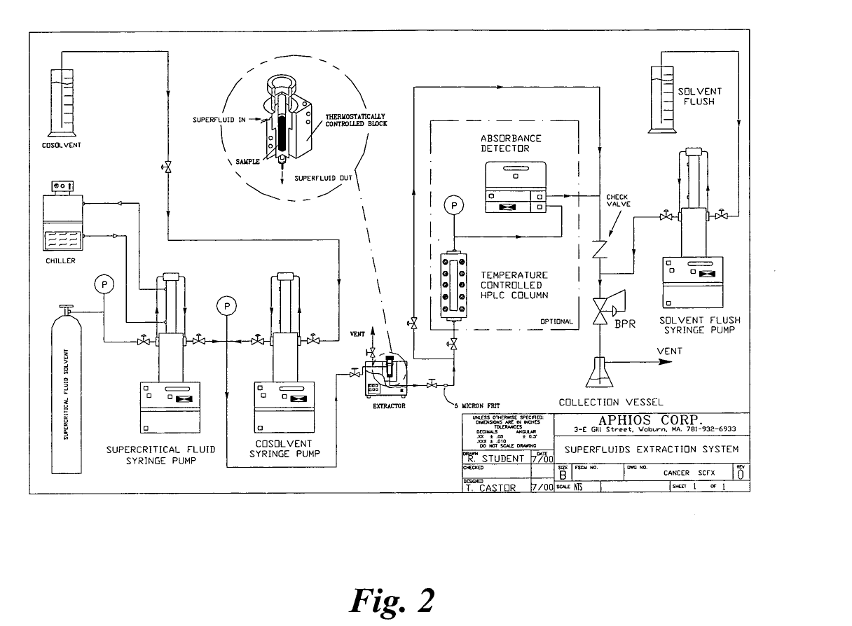

The equipment used to perform the supercritical fluid extraction and purification experiments is shown in FIG. 2. FIG. 1 of U.S. Pat. No. 5,750,709, May 12, 1998 is incorporated as a reference. The apparatus, shown in FIG. 2, is be divided into 4 major subsystems: delivery, extraction, letdown and chromatographic. The delivery system consists of two Model 260-D syringe pumps (Isco, Lincoln, Nebr.) for the supercritical fluid and the cosolvent. The two pumps are operated via a controller that can finely proportionate the delivery of the supercritical fluid and cosolvent on the basis of volume percent while maintaining delivery pressure and flowrate. The system has the capability to run programmed solvent composition gradients. The extraction system shown is an Isco Model SFX 2-10 extraction unit—a dual well, high-pressure device that allows precise and accurate temperature control of both the sample and the supercritical fluid.

Various letdown systems can be employed in the proposed experiments. For example, excellent pressure or flow control can be obtained with a Tescom Model 26-1722 backpressure regulator. The exiting SFS stream containing extracted materials will in some cases be bubbled through methanol, ethanol or acetone; in others, the SFS mixture will be exhausted into a depressurization chamber followed by a low temperature trap. The disengaged SFS will be vented.

Alternatively, experiments that employ chromatographic deposition prior to letdown were used with an HPLC column positioned inline after the SFS extraction step and before the final pressure letdown system. Heaters and insulation are added to the HPLC column to allow better control of the deposition and chromatographic steps. An online variable wavelength UV short path length detector may be included to allow online monitoring of extraction progress and to facilitate the exploration of various extraction conditions.

We investigated the use of carbon dioxide, nitrous oxide, ethylene, ethane, propane and chlorodifluoromethane (Freon-22) as possible supercritical fluid solvents. With the exception of propane and Freon-22, these fluids all have critical temperatures (T_{c}) near ambient. Nitrous oxide and Freon-22 have some polarity while carbon dioxide and ethylene are essentially non-polar. Carbon dioxide, which has a very modest critical point (31° C. and 1,070 psia) is an excellent candidate since it is inexpensive, non-toxic, non-flammable, and environmentally acceptable. Supercritical carbon dioxide has a density of 0.74 gm/cc at a pressure of 2,000 psia and a temperature of 40° C. At and around these conditions, CO₂ behaves like an organic solvent with solubilization characteristics of a liquid and the permeabilization characteristics of a gas.

Near-critical propane may be very effective in solubilizing cannabinoids because of its structure. Near-critical propane is very slightly polar, having a dipole moment of 0.084 Debyes—a factor that may also contribute to its solvation selectivity. Mixtures of near-critical propane and carbon dioxide can also be evaluated to see if propane's flammability limit can be reduced while maintaining or even improving cannabinoid solubilization. Carbon dioxide, with a dipole moment of zero Debyes, may reduce the polarity of near-critical propane and its solvation capacity. We can utilize a 1:4 mixture of carbon dioxide and propane to fractionate the Cannabis biomass; propane will be used as a “high volatility entraining” agent. SFS fractionation experiments with ethylene and ethane can be used to define the effect of hydrocarbon chain length. Ethylene may be of special interest because its low critical temperature (9.6 C) may allow for improved product quality. Freon-22, which has a dipole moment of 1.4 Debyes, can be evaluated in order to define the impact of polarity on the solubilization of cannabinoids.

SFS has additional degrees of freedom over a conventional organic solvent in that their solvation capacities can be readily adjusted by changing density (via changes in temperature and/or pressure), and selectivity can be altered by the type and concentration of entrainers or cosolvents. It is possible to enhance the affinity of supercritical CO₂ for natural pharmaceuticals such as paclitaxel (Taxol) and bryostatin by adding a “low volatility agent” or cosolvent such as an alcohol. Thus, the ability to use cosolvents is an important feature of the experimental apparatus.

Cosolvents—ethanol, methanol, and acetone—can be used to enhance the affinity of the supercritical fluids for the more polar bioactive components of interest. These cosolvents are selected based on structure, polarity and molecular size. Entrainers or cosolvents with carbon dioxide and the other supercritical fluids were evaluated in order to enhance their extraction selectivities. The solvation power of such mixtures can be readily varied by adjustment of pressure, temperature and/or the ratio of supercritical fluid to entrainer. The variation of these parameters was also utilized to selectively precipitate different solute fractions onto the head of an HPLC column for higher resolution chromatographic purification.

Aside from solvent composition, solubilization temperature and pressure must be specified. Related research provides some guidelines for setting these conditions. Supercritical fluids have already been used to extract a wide variety of hydrophobic compounds, some of which have structure similar to those of interest here, e.g. coffee is commercially decaffeinated by selective extraction with supercritical carbon dioxide.

As an example of SFS fractionation/extraction experiment, dried Cannabis is first extracted with supercritical carbon dioxide at 2,000 psig and 40° C. At these conditions, CO₂ has a density of 0.74 g/ml, and solvating characteristics similar to that of hexane. Extraction with CO₂ under these conditions will remove non-polar compounds such as waxes, lipids and possibly Δ9-THC and mixed cannabinoids. Polar compounds will not be extracted. To extract polar compounds, a small quantity of cosolvent such as methanol or acetone is added to the supercritical CO₂. This cosolvent is added gradually and compounds of increasing polarity are extracted. Cannabis sativa biomass is fractionated based on polarity and Hildebrand solubility parameter. Fractions are taken and analyzed. After the experiment is completed, the residue biomass is conventionally extracted and analyzed.

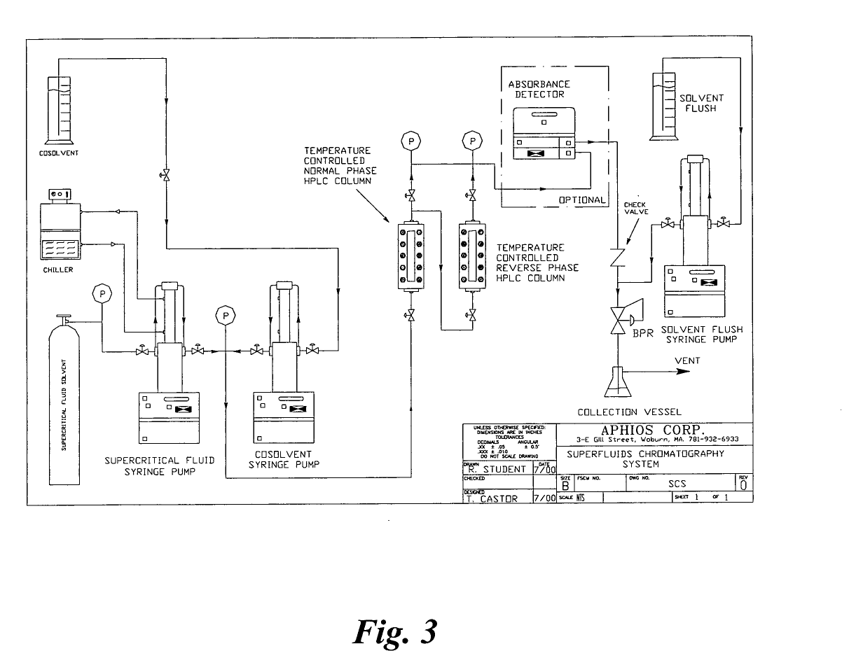

The best conditions for the SFS chromatographic purification of Δ9-THC was established in the experimental apparatus shown in FIG. 3.

Supercritical fluid chromatography is used with packed HPLC-type columns. The low solvent viscosities (supercritical fluids have gas-like properties) allow for efficient operation at high flow velocities yielding separation speed and resolution comparable to that attainable by gas chromatography. Pre-programmed changes in pressure (or of density) can readily be used to optimize separations. Via external computer control, four gradient modes are possible: multilinear pressure, multilinear density, asymptotic pressure and asymptotic density. This apparatus also allows the use of SFS binary gradient systems.

For the chromatography step, as shown in FIG. 3, the extraction unit is taken offline and a reverse phase column installed. A SFS mixture is passed through the normal phase column to elute the materials loaded during the previous extraction step. As the band of compounds of interest such as cannabinoids is about to elute (as can be known from the online absorbance detector), the flow from the normal phase column will be switched to pass through the reverse phase column. After allowing sufficient time for this band to reach the reverse phase column, flow is again diverted to bypass this column. Once the normal phase column has been cleared, flow is directed back to the reverse phase column at altered conditions (temperature, pressure and/or composition) to elute purified fractions.

Deposited cannabinoids and other bioactives will be chromatographically eluted from the silica column using a step gradient of supercritical carbon dioxide and acetone as the polar entrainer. It is of interest to utilize the same co-solvent for both extraction and purification since this would simplify the design and operation of a Cannabis sativa manufacturing facility. SFS chromatography may provide a striking advantage over organic phase chromatography in that the elutant of the silica column can be deposited directly onto the head of the reversed phase column.

Normal phase SFS chromatography gives significant advantages over traditional chromatography. Replacement of hexane with carbon dioxide results in an environmentally friendly mobile phase with considerably reduced viscosity. Thus, with the SFS mobile phase, it may be possible to use much longer chromatographic columns to obtain higher plate counts and superior separations. Fractions obtained from the supercritical fluid columns are of higher purity than those obtained using conventional columns.

Preliminary experiments were conducted on a Δ9-THC-rich side cut in order to define the best SFS mobile phase and column conditions required to give good separation efficiencies and a high number of theoretical plates. Even though the organic gradient and isocratic mobile phases can be readily emulated, the characteristics of the SFS system are significantly and positively impacted by altering the solubilization characteristics of this mobile phase through pressure programming. From an engineering standpoint, one would prefer to deposit the SFS extract on the head of a normal phase chromatographic column and then proceed with SFS chromatography. Depending on the response of Cannabis sativa to SFS extraction, this approach may or may not be possible. If the level of cosolvent required for selective extraction is too high, then the cosolvent may deactivate the chromatographic column. If direct coupling to the normal phase chromatographic (silica) column is not feasible, then the SFS extract will be extended on silica to remove the cosolvent and packed into a guard column that will be coupled to the chromatographic columns. SFS chromatography will then proceed without interference from the cosolvent.

If direct coupling of the normal phase to the reverse phase column (C18) is not possible, high purity fractions obtained from SFS normal phase chromatography will be combined, extended onto C18, and chromatographed on C18 using conventional preparative equipment with a water/methanol gradient. This reversed-phase purification will result in nearly pure Δ9-THC.

The Δ9-THC-rich SFS fractions will be further purified, if necessary, by conventional reverse phase chromatography and/or SFS normal phase chromatography. Celite will be added to the Δ9-THC-rich fractions and excess cosolvent (e.g., acetone or methanol) will be removed by rotary evaporation under vacuum. The celite, coated with Δ9-THC and other impurities, will be then packed into a loading column that will be connected to a reverse phase column (either C18 or CG-71). Δ9-THC will be separated from impurities using a methanol/water or acetonitrile/water gradient. Elutants will be periodically sampled and assayed by HPLC. Recycle of the chromatography mobile phase will be used during product elution to minimize mobile phase usage.

Alternatively, if necessary, the Δ9-THC-rich fractions will be purified by normal phase chromatography utilizing a SFS CO₂/acetone mobile phase. Acetone is a preferred to methanol as a cosolvent in this case because methanol, at high enough concentrations, will deactivate the silica columns. The choice between reverse-phase utilizing a methanol-water system versus a normal phase system utilizing a SFS CO₂/acetone mobile phase will be governed by impurity profiles. Indeed, in certain cases, both systems may be required.

### EXAMPLES

### Example 1

### Cannabis Raw Materials

Under DEA Schedule I license (No. RC0288058), two 250-gram shipments of high potency, bulk marijuana biomass were requested from the National Institute of Drug Abuse (NIDA). Both shipments were provided by the Research Triangle Institute (RTI) International, Research Triangle Park, N.C. under contract with NIDA. The first batch (RTI Batch No. 8976-1001-26/University of Mississippi Batch No. 1182/1186) was received and logged in at Aphios on Dec. 17, 2002. This batch was measured by the University of Mississippi's Thad Cochran National Center for the Natural Products Research to have a Δ9-THC content of 8.98%. Table 1 lists the concentration of other cannabinoids per the provided Certificate of Analysis (Dec. 2, 2002). Table 1 also lists HPLC measurements of heat-treated biomass made by Aphios' chemists; the methods and data are detailed below. The biomass was stored in a locked safe at room temperature when not in use. All usage was recorded on a log sheet that was also stored in the same safe.

The second shipment, also from the same batch of high potency marijuana from the U. of Mississippi, was received and logged in at Aphios on Aug. 14, 2003. The second shipment was measured by RTI on Jul. 29, 2003 to have a Δ9-THC content of 6.12% by GLC analysis. Table 1 lists the concentration of other cannabinoids per RTI's analysis of Jul. 29, 2003, compared to Aphios' HPLC analysis of the heat-treated biomass on Sep. 2, 2003.

The Δ9-THC content of the marijuana biomass decreased about 32% over an 8-month period at room temperature storage conditions at RTI, suggesting that the biomass should be stored at refrigerated temperatures when not being processed.

TABLE 1
Cannabinoid Content of Marijuana Biomass Raw Materials

| | Composition of First Marijuana | | Composition of Second Marijuana | |
|---|---|---|---|---|
| | Shipment (%) | | Shipment (%) | |
| | U. of Mississippi | Aphios | RTI | Aphios |
| Cannabinoid | (Dec. 17, 2002) | (Jan. 13, 2003) | (Jul. 29, 2003) | (Sep. 02, 2003) |
| Δ9-THC | 8.98 | 8.44 | 6.12 | 6.20 |
| CBD | 0.31 | 0.87 | 0.19 | NM |
| CBN | 0.17 | 0.33 | 0.16 | NM |
| Δ8-THC | NR | 0.00 | NR | NM |
| Δ9-THCA | NR | 0.17 | NR | 2.06 |
| THCV | 0.09 | NM | NR | NM |
| CBC | 0.19 | NM | NR | NM |
| CBG | 0.44 | NM | NR | NM |
| Moisture | NR | 7.41 | 9.63 | NM |

Δ9-THC—Δ9-tetrahydrocannabinol
Δ9-THCA—Δ9-tetrahydrocannabinolic acid
CBD—cannabidiol
THCV—tetrahydrocannabivarin
CBN—cannabinol
CBC—cannabicbromene
Δ8-THC—Δ8-tetrahydrocannabinol
CBG—cannabigerol
NR—Not Reported
NM—Not Measured

### Example 2

### Cannabinoid Standards

Four (4) standards were purchased for chromatographic assay. They were all purchased at certified concentrations of 1 mg/ml in methanol and transported at ambient atmosphere in sealed glass vials. The standards are as follows:

### 1. Cannabidiol, C₂₁H₃₀O₂, MW=314.47, (99.9%) [Alltech]

### 2. Δ-8-THC, C₂₁H₃₀O₂, MW=314.45, (90.0%) [Alltech]

### 3. Cannabinol, C₂₁H₂₆O₂, MW=310.42, (98.9%) [Alltech]

### 4. Δ-9-THC, C₂₁H₃₀O₂, MW=314.45, (97%) [ChromaDex, Santa Ana, Calif.]

Under Aphios' DEA Schedule I license, we requested and obtained 5 ml of 50 mg/ml Δ9-THC in absolute (100%) ethanol for use as an analytical standard.

### Example 3

### HPLC Analysis

Two (2) HPLC methods were evaluated for the analysis of Δ9-THC, Δ-8-THC, CBN, CBD and Δ9-THCA. Since Δ9-THCA is the precursor of Δ9-THC via decarboxylation (heat) and CBN is the degradation (oxidative) product of Δ9-THC, both compounds must be resolved by the chromatography system. CBD is not psychotomimetic in pure form although it does have sedative, analgesic and antibiotic properties. CBD can contribute to the psychotropic effect by interacting with Δ9-THC to potentiate or antagonize certain qualities of this effect. Δ9-THC is the main psychotomimetic (mind-bending) compound of Cannabis. Δ-8-THC is slightly less active and is reported in low concentrations, less than 1% of Δ9-THC, and may be an artifact of the extraction/analysis process.

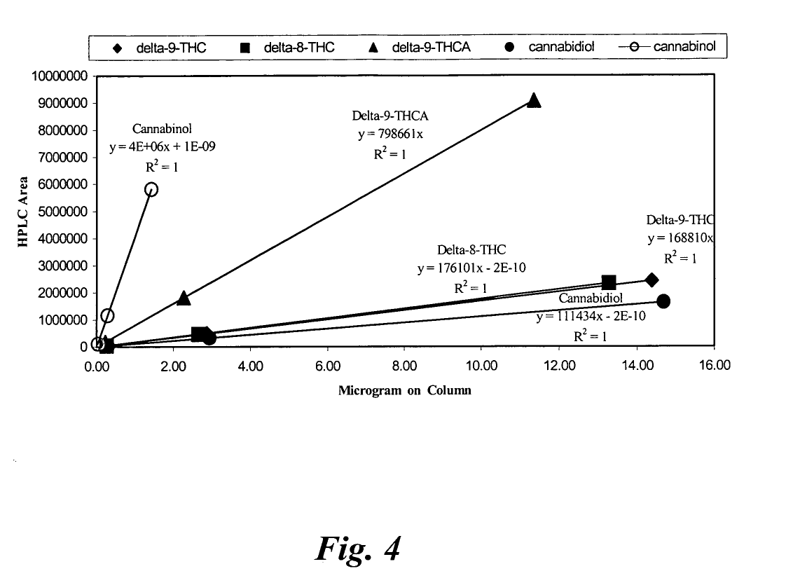

The two HPLC methods evaluated were: (1) a gradient system utilizing a modified Phenomenex method; and (2) an isocratic system that is a modification of the Maripharm, Rotterdam, Netherlands method. The latter system was selected based on peak separation and product purities. This isocratic method utilized a Phenomenex Luna 3 μm C18 column (5 cm×4.6 mm) with a pre-column at 25° C. The mobile phase, at 1.0 ml/min, consisted of 78% methanol:22% water containing 1% acetic acid. Absorbance was monitored by a Waters Photodiode Array (PDA) detector, Model 996, and measured at 285 nm and 230 nm. The analytical HPLC system included a Waters 717 Autosampler, 600E System Controller and a Waters Dual-Piston High Pressure HPLC pump, Model No. 600, driven by a Pentium 4 Personal Computer and controlled by Waters Millennium 4.0 software. Temperature of the HPLC column was controlled by an Eppendorf CH-30 column heater. This isocratic system was utilized to analyze the Cannabis biomass and experiments MAJ-1 to MAJ-22. In order to reduce run time for Phenomenex Luna 5 and 10 μm C18 columns, the mobile phase was changed to 80% acetonitrile:20% water containing 0.1% acetic acid at a flowrate or 2.0 ml/min and a column temperature of 30° C. with absorbance measurement at 285 nm. This isocratic system was utilized to analyze fractions from experiments MAJB-1 to MAJB-10. Also, using this isocratic system, a second HPLC system (Isco) was utilized to monitor the column chromatography utilized in the downstream purification. Representative regression curves of cannabinoid standards are shown in FIG. 4.

### Example 4

### HPLC Analysis of Cannabis Biomass

0.5 g of grounded Cannabis biomass was extracted four times with 20 ml of methanol:chloroform::90:10 by sonication. The extracts were filtered and combined in a 100 ml Class A volumetric flask. The biomass residue was then rinsed with 10 ml of the extraction solvent and added to the flask. The flask was adjusted to volume with the extraction solvent. This extract was assayed on HPLC using the isocratic system and total solids were determined. The procedure was conducted for unheated biomass and for Cannabis biomass that was oven dried at 100° C. for 2.5 hours. 0.5 ml of both extracts was transferred into 1.0 ml reaction vials. The vials were then heated for 2.5 hours at 100° C. using a heating block. The heated extracts were assayed on HPLC using the isocratic system. The extracted biomass was air dried under the hood and returned to the safe for inventory/usage tracking. These results are summarized in Table 2.

TABLE 2
Cannabinoid Content of Heated and Unheated Cannabis sativa and its
Extracts

| | Unheated Cannabis Biomass | | Heated Cannabis Biomass | |
|---|---|---|---|---|
| | Unheated | | Unheated | |
| | Extract | Heated Extract | Extract | Heated Extract |
| Cannabinoid | Cannabinoid Concentration (%) | | | |
| Δ9-THC | 0.93 | 8.44 | 8.43 | 8.47 |
| Δ8-THC | 0.00 | 0.00 | 0.00 | 0.00 |
| Cannabidiol | 0.15 | 0.87 | 0.87 | 0.67 |
| Cannabinol | 0.09 | 0.33 | 0.33 | 0.35 |
| Δ9-THCA | 10.7* | 0.17 | 0.17 | 0.09 |

*estimated based on stoichometrically conversion to Δ9-THC

### Example 5

### SFS Extraction of Untreated Cannabis Sativa Biomass

To prepare a sample, the biomass was first dried in a convective oven at 45° C. for 18 hours and then ground into a fine powder (approximately 40 mesh). The dried powder was transferred to a 10 ml stainless-steel extraction cartridge, after which the cartridge was sealed. After loading a cartridge on the cartridge holder, the biomass was extracted at pre-specified conditions of temperature and pressure for 1½ hours with fractions taken every 30 minutes. After completion of the run, the spent biomass was recovered and returned to inventory control in a locked safe. The extracts were dissolved in methanol and were bought to a known volume with methanol. A portion of this was assayed by HPLC and a second portion was evaporated to dryness for determination of the solids content.

A total of 17 experiments with different SFS at different pressures and temperatures were conducted on unheated Cannabis sativa, yielding 52 samples for HPLC analyses. Most of the extractions were conducted with neat supercritical or near-critical fluids without a cosolvent. One experiment was conducted with a sequential extraction/fractionation procedure in which the cosolvent methanol concentration was increased from 0% in neat supercritical CO₂ at 3,000 psig and 40° C. to 40% methanol in 5% to 10% increments, each step being 30 min at a combined flow rate of 2.0 ml/min. This experiment yielded 5 fractions, which were transferred to the separate pre-weighed glass vials.

### Example 6

### Impact of Pressure

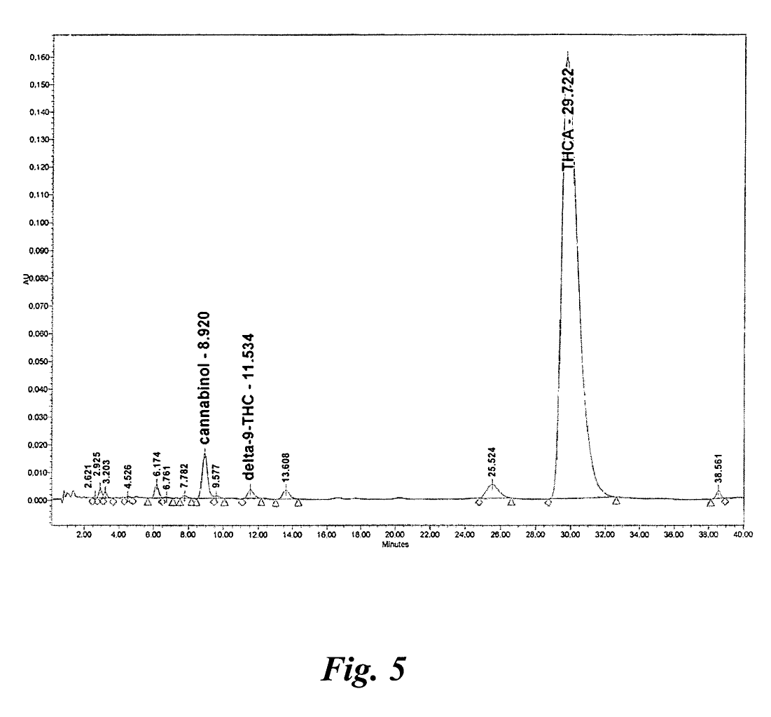

Data in Table 3 for the SFS extraction of unheated Cannabis sativa indicates that isolation of Δ9-THC and Δ9-THCA is near optimum at 4,000 psig for near-critical CO₂ at 25° C. Yield is based on the percentage of Δ9-THC and Δ9-THC recovered in the biomass relative to the analysis listed in Table 2 (10.7% Δ9-THCA and 0.93% Δ9-THC). Absolute purity of Δ9-THC is based on the standard obtained from Chromodex, Santa Ana, Calif.; and absolute purity of Δ9-THCA is estimated based on its conversion to Δ9-THC. The best extraction conditions were established in MAJ-08 at a pressure of 4,000 psig and a temperature of 25 C; an HPLC chromatogram of MAJ-8 is shown as FIG. 5.

TABLE 3
SFS Extraction of Untreated Cannabis Sativa Biomass
as a Function of Pressure at a Temperature of 25° C.

| | | | | | Δ9-THC | | Δ9-THCA | |
|---|---|---|---|---|---|---|---|---|
| | SFS Extraction Parameters | | | | | Absolute | | Absolute |
| | | P | T | | Yield | Purity | Yield | Purity |
| Expt. No. | Fluid | (psig) | (° C.) | Cosolvent | (%) | (%) | (%) | (%) |
| MAJ-05 | CO₂ | 1000 | 25 | none | 66.7 | 21.49 | 13.9 | 51.55 |
| MAJ-06 | CO₂ | 2000 | 25 | none | 80.6 | 14.83 | 27.7 | 58.47 |
| MAJ-07 | CO₂ | 3000 | 25 | none | 83.9 | 10.45 | 44.2 | 62.95 |
| MAJ-08 | CO₂ | 4000 | 25 | none | 100.0 | 7.61 | 88.2 | 77.20 |
| MAJ-10 | CO₂ | 5000 | 25 | none | 57.0 | 6.23 | 50.5 | 63.93 |
| MAJ-14 | CO₂ | 5000 | 25 | none | 52.7 | 7.64 | 37.7 | 62.40 |

### Example 7

### Impact of Temperature

The effect of temperature on the SFS extraction of untreated Cannabis sativa is shown in Table 4. At 5,000 psig, the best extraction condition appears to be around 35° C. for supercritical CO₂ over the narrow temperature range examined. The yields and absolute purities of Δ9-THC and Δ9-THCA are, however, not as good as those obtained at 4,000 psig for near-critical CO₂ at 25° C.

TABLE 4
SFS Extraction of Untreated Cannabis Sativa Biomass
as a Function of Temperature

| | | | | | Δ9-THC | | Δ9-THCA | |
|---|---|---|---|---|---|---|---|---|
| | SFS Extraction Parameters | | | | | Absolute | | Absolute |
| | | P | T | | Yield | Purity | Yield | Purity |
| Expt. No. | Fluid | (psig) | (° C.) | Cosolvent | (%) | (%) | (%) | (%) |
| MAJ-10 | CO₂ | 5000 | 25 | none | 57.0 | 6.23 | 50.5 | 63.93 |
| MAJ-14 | CO₂ | 5000 | 25 | none | 52.7 | 7.64 | 37.7 | 62.40 |
| MAJ-11 | CO₂ | 5000 | 35 | none | 67.7 | 5.76 | 62.1 | 60.98 |
| MAJ-12 | CO₂ | 5000 | 45 | none | 53.8 | 5.65 | 45.9 | 55.81 |

### Example 8

### Impact of SFS

The effect of different SFS on the extraction of untreated Cannabis sativa is shown in Table 5 for a pressure of 5,000 psig and a temperature of 25° C.

TABLE 5
SFS Extraction of Untreated Cannabis Sativa Biomass
as a Function of Different SFS

| | | | | | Δ9-THC | | Δ9-THCA | |
|---|---|---|---|---|---|---|---|---|
| | SFS Extraction Parameters | | | | | Absolute | | Absolute |
| | | P | T | | Yield | Purity | Yield | Purity |
| Expt. No. | Fluid | (psig) | (° C.) | Cosolvent | (%) | (%) | (%) | (%) |
| MAJ-14 | CO₂ | 5000 | 25 | none | 52.7 | 7.64 | 37.7 | 62.40 |
| MAJ-15 | Freon-22 | 5000 | 25 | none | 33.3 | 5.00 | 24.1 | 42.15 |
| MAJ-16 | Freon-23 | 5000 | 25 | none | 35.5 | 23.53 | 4.8 | 36.06 |
| MAJ-17 | Propane | 5000 | 25 | none | 79.6 | 6.79 | 71.3 | 70.02 |

At 5,000 psig and 25° C., near-critical propane provides the highest yields of Δ9-THC and Δ9-THCA of the fluids tested. Near-critical propane is only very slightly polar, having a dipole moment of 0.084 Debyes—a factor that may also contribute to its solvation selectivity of certain complex organic molecules. The yields and absolute purities of Δ9-THC and Δ9-THCA are, however, not as good as those obtained at 4,000 psig for near-critical CO₂ at 25° C.

Freon-22 (dichlorofluoromethane) and Freon-23 (trifluoromethane), with their large dipole moments, were tested to determine their selective extraction efficiencies relative to carbon dioxide. Freon-23 was of particular interest since it is non-chlorinated. These fluids were not as effective as CO₂ or propane and were thus removed from further consideration. Propane was also eliminated since it is more expensive, toxic and flammable than carbon dioxide. Current plans are to recover and recycle the SFS CO₂ and any cosolvent utilized in order to minimize operating costs and environmental impact.

### Example 9

### Impact of Cosolvent

The SFS CO₂ fractionation of untreated Cannabis Sativa biomass as a function of different cosolvent concentrations at 25° C. and 5,000 psig is summarized in Table 6. For these sub-optimal conditions for CO₂, most of the Δ9-THC and Δ9-THCA are extracted in the first fraction (0% methanol); however, an additional 20% of Δ9-THC and 25% Δ9-THCA were recovered in the second fraction (5% methanol), increasing the overall yield. Under optimal conditions for CO₂, a cosolvent may not be required for Δ9-THC as shown in Table 3. A cosolvent may, however, be useful for the recovery of Δ9-THCA, which is slightly more polar than Δ9-THC.

TABLE 6
SFS CO₂ Extraction of Untreated Cannabis Sativa Biomass as a Function of
Different Cosolvent Concentrations at Temperature of 25° C. and Pressure of 5,000 psig

| | | | | | Δ9-THC | | Δ9-THCA | |
|---|---|---|---|---|---|---|---|---|
| | SFS Extraction Parameters | | | | | Absolute | | Absolute |
| | | P | T | | Yield | Purity | Yield | Purity |
| Expt. No. | Fluid | (psig) | (° C.) | Cosolvent | (%) | (%) | (%) | (%) |

| | | | | | | | | |
|---|---|---|---|---|---|---|---|---|
| MAJ-09-01 | CO₂ | 5000 | 25 | 0% methanol | 43.0 | 5.40 | 42.3 | 61.13 |
| MAJ-09-02 | CO₂ | 5000 | 25 | 5% methanol | 8.6 | 4.28 | 10.9 | 62.46 |
| MAJ-09-03 | CO₂ | 5000 | 25 | 10% methanol | 2.2 | 2.96 | 2.2 | 44.93 |
| MAJ-09-04 | CO₂ | 5000 | 25 | 20% methanol | 2.2 | 1.94 | 2.5 | 22.24 |
| MAJ-09-05 | CO₂ | 5000 | 25 | 40% methanol | 0.00 | 1.20 | 0.3 | 6.92 |

### Example 10

### SFS Extraction of Heated Cannabis Biomass

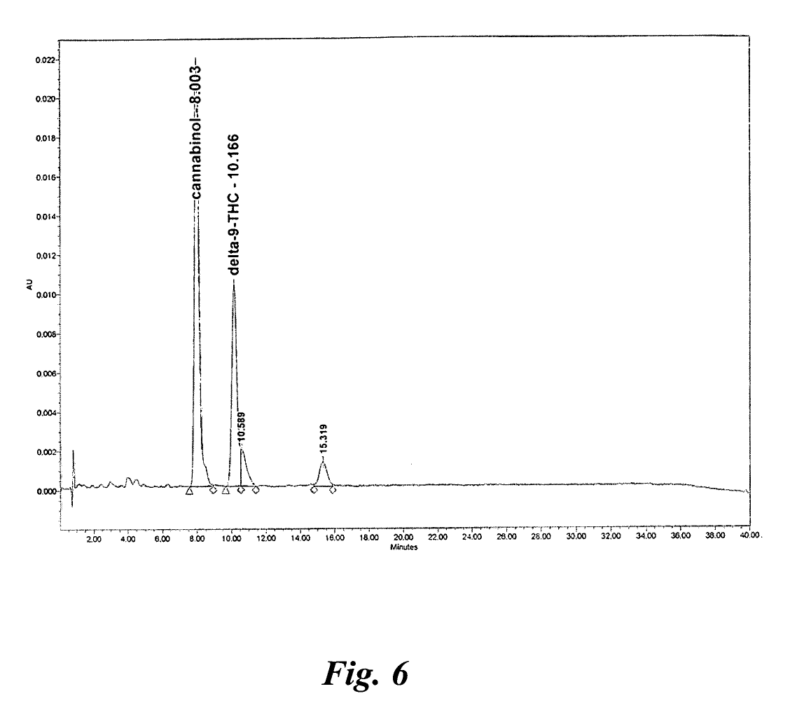

Six (6) SFS extraction experiments were conducted on Cannabis sativa biomass that had been heated at 100° C. for 2.5 hours to convert the Δ9-THCA into Δ9-THC. The results of these experiments are listed in Table 7, in which the yield for Δ9-THC is based on the 8.43% value for the cannabinoid content of heated biomass listed in Table 2. These experiments, conducted as a function of pressure with CO₂ at 25° C., indicates that the best pressure for extracting Δ9-THC is 1,000 psig, resulting in a yield of 70.3% and an absolute purity of 68.5%. An HPLC chromatogram of the primary fraction of MAJ-21 is shown as FIG. 6. As expected for the heat-treated Cannabis biomass, no Δ9-THCA was extracted in any of the experiments conducted on the heat-treated marijuana.

TABLE 7
SFS CO₂ Extraction of Heat-Treated Cannabis Sativa Biomass
as a Function of Pressure at a Temperature of 25° C.

| | | | | | Δ9-THC | | Δ9-THCA | |
|---|---|---|---|---|---|---|---|---|
| | SFS Extraction Parameters | | | | | Absolute | | Absolute |
| | | P | T | | Yield | Purity | Yield | Purity |
| Expt. No. | Fluid | (psig) | (° C.) | Cosolvent | (%) | (%) | (%) | (%) |
| MAJ-18 | CO₂ | 2000 | 25 | none | 30.1 | 28.06 | 0.00 | 0.00 |
| MAJ-19 | CO₂ | 4000 | 25 | none | 68.1 | 59.35 | 0.00 | 0.00 |
| MAJ-20 | CO₂ | 5000 | 55 | none | 62.9 | 56.30 | 0.00 | 0.00 |
| MAJ-21 | CO₂ | 1000 | 25 | none | 70.3 | 68.51 | 0.00 | 0.00 |
| MAJ-22 | CO₂ | 3000 | 25 | none | 61.2 | 59.55 | 0.00 | 0.00 |
| MAJ-23 | CO₂ | 5000 | 25 | none | 60.4 | 56.26 | 0.00 | 0.00 |

### Example 11

### SFS Extraction and Chromatography of Untreated and Heat-Treated Biomass

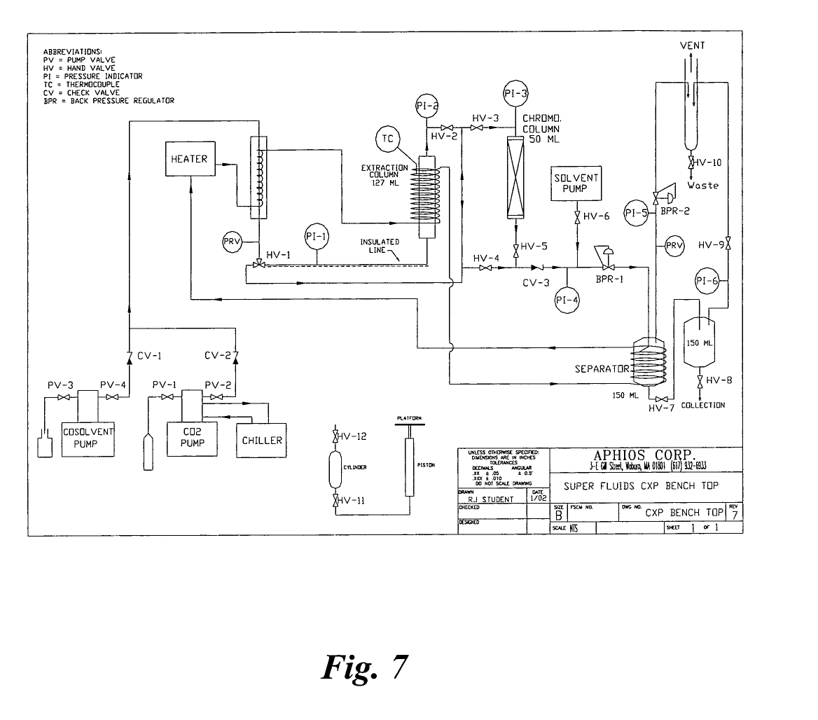

The bench-top apparatus utilized for the SFS extraction and chromatography for Cannabis sativa is shown as FIG. 7. This apparatus consists of a 127 ml high-pressure extraction column and a 50 ml high-pressure chromatographic column with accessory devices for controlling temperature, pressure and flowrate of near-critical, critical and supercritical fluids with or without polar cosolvents. The entire apparatus is rated for 5,000 psig and 100° C. with operating flow-rates of up to 100 ml/min. Each experiment conducted utilized around 40 grams of marijuana, a scale-up factor of ˜16 from the laboratory-scale experiments conducted.

### Example 12

### SFS Extraction and Chromatography of Heat-Treated Cannabis Biomass

Heat-treated Cannabis (100° C. for 2.5 hours) was first extracted in the bench-top SFS-CXP apparatus to evaluate the scale-up from the laboratory-scale extraction apparatus. In this experiment, MAJB-1, 41.35 g of heat-treated marijuana was extracted with near-critical CO₂ at 4,000 psig and 25° C. with a flowrate of 40 ml/min for 1.6 hours. Three fractions were taken, one every 30 minutes. The overall yield of the Δ9-THC target was 90.3% and the absolute purities of Δ9-THC in the three major fractions were 55.6, 68.9 and 71.1%. The overall yield was computed after exhaustively extracting the spent biomass with organic solvents to determine residual Δ9-THC content.

In subsequent experiments, a silica chromatographic column was put on line during the extraction of the heat-treated marijuana with neat CO₂. In MAJB-4, 44.04 g of heat-treated marijuana was extracted with near-critical CO₂ at 4,000 psig and 25° C. at a flowrate of 40 ml/min for 1.4 hours. The SFS extract was continuously loaded onto an activated silica chromatographic column (50 ml, 2.5 cm ID×10 cm long, 25 grams). The flow through fractions were collected, dried, weighed and analyzed by HPLC. After extraction, the extraction column was bypassed and the chromatographic column eluted with a methanol:CO₂ gradient. The gradient started with 0.2% methanol/99.8% CO₂ for 15 minutes at a combined flowrate of 10 ml/min (˜1 extractor volume), and was increased in 0.2% increments of methanol until its concentration was 2% in 98% CO₂. The flow through fractions were collected and analyzed by HPLC.

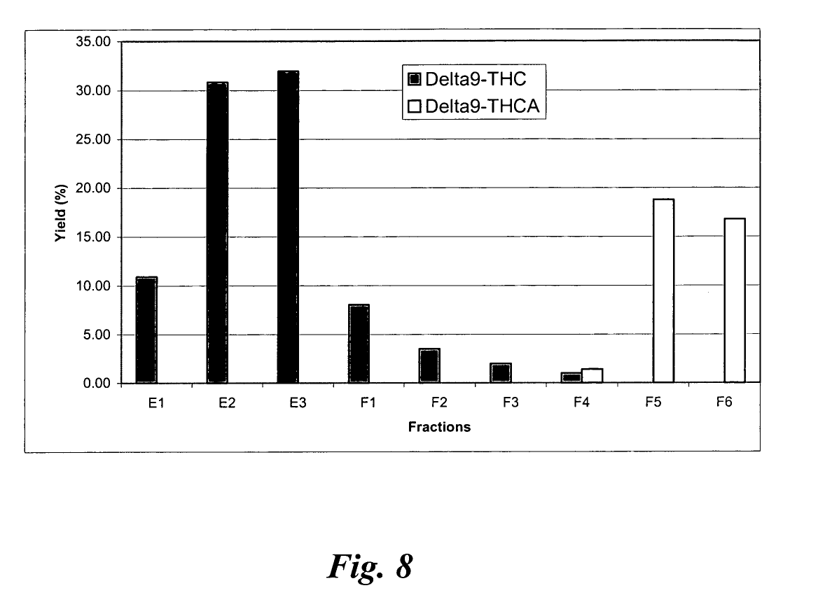

The results of MAJB-4 are shown in FIG. 8. E1, E2 and E3 are the flow through fractions obtained during the extraction step. F1 to F6 are fractions obtained during the chromatographic step; F7-F11, which were free of the target cannabinoids, are not reported The SFS CXP silica chromatography clearly separates Δ9-THC from Δ9-THCA. The absolute purities of the Δ9-THC were 54.1, 65.0 and 68.7% for fractions E1, E2 and E3, respectively. The extraction and elution profiles show good separation between Δ9-THC and Δ9-THCA.

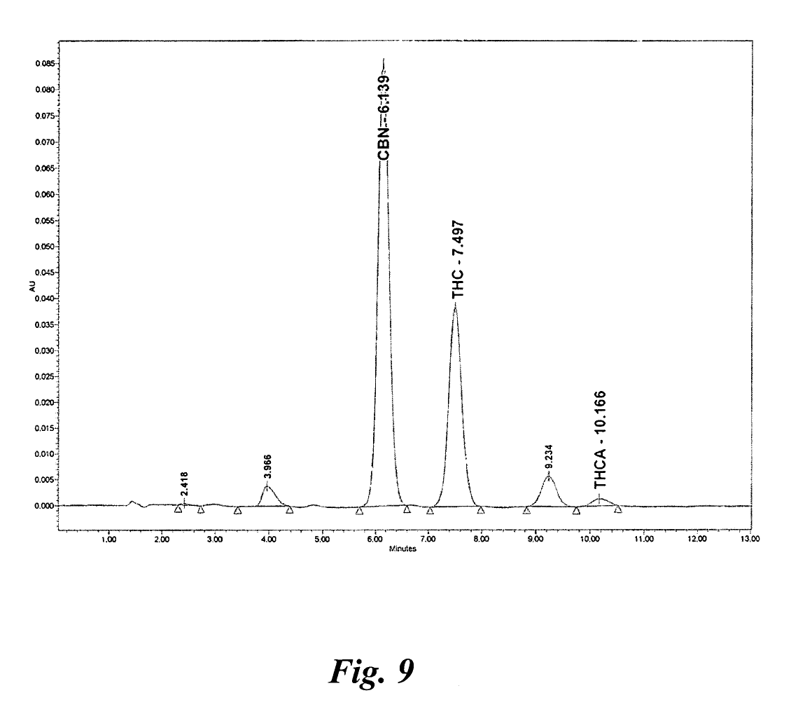

An HPLC chromatogram of the middle extraction fraction, F2, is shown as FIG. 9. In this chromatogram, the other major peak is cannabinol (CBN). The relative mass of CBN to Δ9-THC is about 15-20 time less, even though it appears much greater because of its high UV response factor. CBN is a degradation (oxidative) product of Δ9-THC and is thought to be generated by poor storage and/or processing conditions.

Several other runs were conducted to evaluate the relative amount of silica required to improve the separation efficiency between Δ9-THC and CBN. These experiments did not improve the separation efficiency since they were all conducted with increasing ratios of silica to Cannabis biomass. Since Δ9-THC appears to have some affinity for silica, the ratios can be decreased in further studies.

Use of C18 instead of silica in MAJB-5, all other conditions being the same as MAJB-4, worsened the separation, as Δ9-THCA was obtained in all the flow through extraction step fractions [data not shown]. The overall recovery efficiencies, however, remained around the same (˜85%) as MAJB-4 since all the other run conditions were the same.

TABLE 8
SFS Extraction and Chromatography of Untreated Cannabis Sativa
Biomass At a Pressure of 4,000 psig and Temperature of 25° C.

| | | | | | Δ9-THCA | | |
|---|---|---|---|---|---|---|---|
| | Fluid | Cosolvent | | Column | | | Abs. |
| | CO₂ | Methanol | Time | Vol- | Amount | Yield | Purity |
| Fraction | (%) | (%) | (mins) | umes | (mg) | (%) | (%) |
| E1 | 100 | 0 | 28.6 | 9 | 0.0 | 0.0 | 0.0 |
| E2 | 100 | 0 | 28.6 | 9 | 0.0 | 0.0 | 0.0 |
| E3 | 100 | 0 | 28.6 | 9 | 0.0 | 0.0 | 0.0 |
| E4 | 100 | 0 | 28.6 | 9 | 35.9 | 1.39 | 33.9 |
| E5 | 100 | 0 | 28.6 | 9 | 139.4 | 5.41 | 60.1 |
| E6 | 100 | 0 | 28.6 | 9 | 196.7 | 7.64 | 74.5 |
| F1 | 99.8 | 0.2 | 15 | 3 | 74.5 | 2.89 | 58.7 |
| F2 | 99.6 | 0.4 | 15 | 3 | 108.6 | 4.22 | 68.7 |
| F3 | 99.4 | 0.6 | 15 | 3 | 99.9 | 3.88 | 78.7 |
| F4 | 99.2 | 0.8 | 15 | 3 | 100.3 | 3.89 | 78.4 |
| F5 | 99.0 | 1.0 | 15 | 3 | 183.4 | 7.12 | 78.7 |
| F6 | 98.8 | 1.2 | 15 | 3 | 280.4 | 10.89 | 64.8 |
| F7 | 98.6 | 1.4 | 15 | 3 | 411.1 | 15.96 | 77.6 |
| F8 | 98.4 | 1.6 | 15 | 3 | 343.0 | 13.32 | 83.3 |
| F9 | 98.2 | 1.8 | 15 | 3 | 75.3 | 2.92 | 40.3 |
| F10 | 98.0 | 2.0 | 15 | 3 | 5.4 | 0.21 | 7.5 |
| Totals | | | 321 | 57 | 2053.9 | 79.74 | |
| Residue | | | | | 522.0 | | |

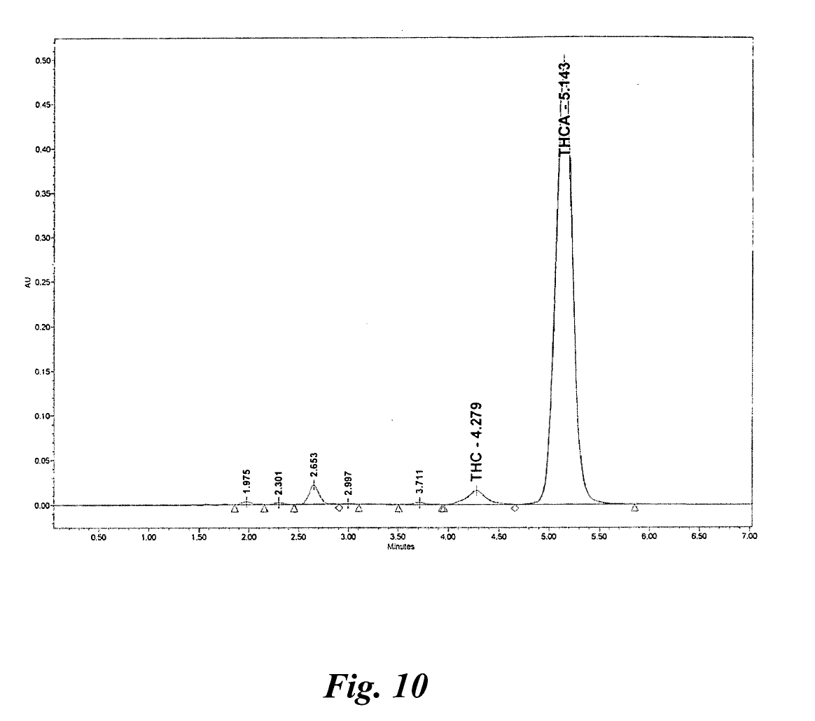

In Table 8, fractions E1-E6 were collected after flowing the neat CO₂ Cannabis extract though a silica column, as previously described. The mobile phase was then changed to a CO₂:methanol gradient, the extraction column bypassed, the chromatographic column eluted at a lower flow-rate and fractions F1 to F10 collected. The absolute purities of Δ9-THCA in the major fractions ranged from 59% to 83%. An HPLC chromatogram of fraction MAJB-3-F8, is shown as FIG. 10.

The overall yield of MAJB-3 could have been increased beyond 80% by either increasing the extraction time with neat CO₂ and/or by adding a cosolvent step, as suggested by the data in Table 6. The latter was demonstrated in MAJB-9 by following the neat CO₂ extractions steps with a CO₂: methanol::95:5 step. MAJB-9 resulted in an overall Δ9-THCA yield of 92.6%. In MAJB-10, the extraction step was increased by 50% over MAJB-3. The Δ9-THCA-rich CO₂ stream was routed through the silica column and eluted as previously described. The Cannabis biomass was then extracted with 5% methanol:95% CO₂. MAJB-10 resulted in an overall Δ9-THCA yield of 85.4% with high absolute purities ranging from 59% to 82%. These results suggest that a combination of increased extraction with neat CO₂ and use of a small amount of cosolvent (5% methanol or less) will be optimum for isolating Δ9-THCA.

### Example 13

### Downstream Purification of Δ9-THC and Δ9-THCA

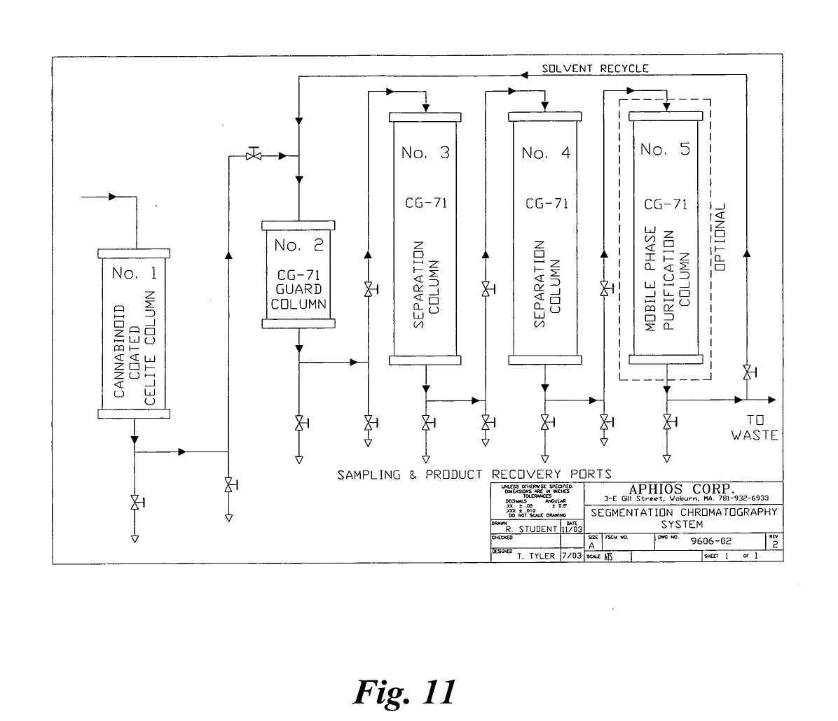

The downstream purifications of both Δ9-THC and Δ9-THCA utilized a 5-column, reversed-phase chromatographic system shown as FIG. 11. This system consists of a loading column (#1), a guard column (#2), two separation columns (#3 and #4) for isolating closely related elutants and a mobile phase solvent purification column (#5) for removing impurities prior to recycling the mobile phase.

As the target compounds move from column 1 to columns 2, 3 and 4, fast-movers move to column 5. Column 5 is used to trap the fast movers, thus providing fresh solvent for the recycle. Eventually, the target compounds are moved to column 4 leaving the faster-moving compounds on column 5 and the slower-moving compounds on columns 1, 2, and 3. Column 4 is eluted to yield purified fractions of the target compounds.

### Example 14

### Column Segmentation Chromatography Utilizing CG71

Amberchrome CG-71 is a polymeric chromatographic stationary phase supplied by Tosoh Corporation, Tokyo, Japan. It is polymethylmethylacyralate and can be obtained as particles from 35 to 120 microns in diameter. It has selectivity similar to that of C18 and has a greater loading capacity than C18 since the entire particle is active, not just the surface.

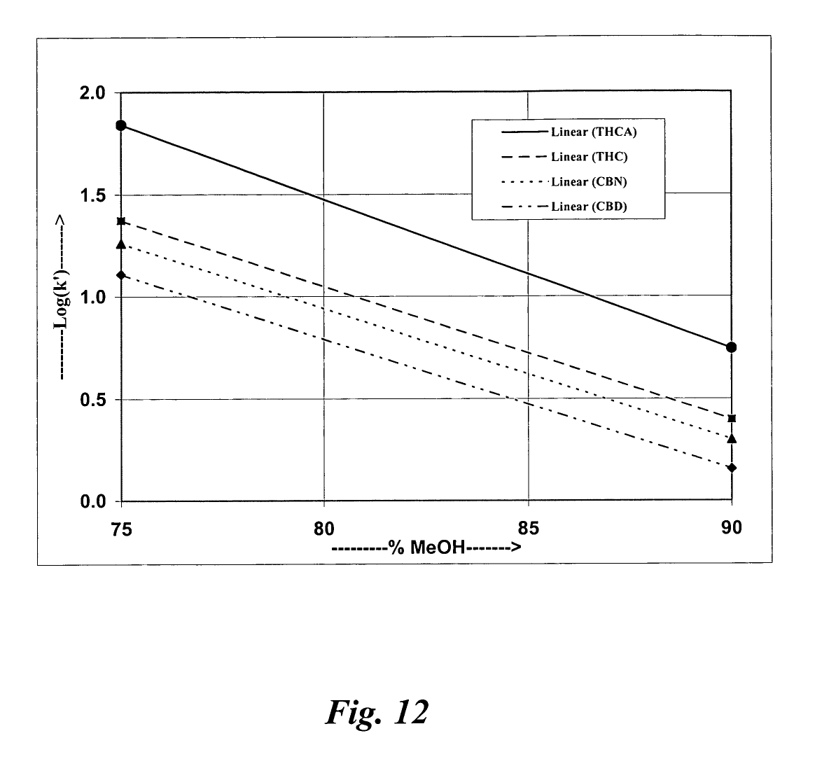

The mobility of various cannabinoid compounds on a CG-71 column was utilized to design their separation on CG-71. To do this we needed an HPLC column with CG-71 packing. Unfortunately, this was not available so a Phenomenex Luna 10 micron C18 column was used as a substitute. The selectivity, k, was expected to be similar for some compounds and dissimilar for others. A standard plot of log (k) versus % methanol, as shown in FIG. 12, was made. The plot shows that Δ9-THCA moves slowly and is well separated from the other three major components. On C18 the elution order was found to be CBD, CBN, Δ9-THC and Δ9-THCA.

The retention time data gave a good linear response (y=mx+b) and the “m” and “b” values were determined for the four components. Standard chromatographic calculations were then made which predicted the number of centimeters that a component would move down the column. The equations were set up in an Excel spreadsheet. Once the spreadsheet has appropriate “m” and “b” values from the graph of Log (k) vs % methanol, different values can be entered for % methanol, flow-rate and elution time so that the migration distance can be determined under different conditions. When the system was set up with 5-cm CG-71 columns, it was found that: (a) Δ9-THCA moved as predicted; (b) Δ9-THC moved as had been predicted for CBN; (c) CBN moved slightly slower than the Δ9-THC.

Fractions from the SuperFluids™ CXP of heat-treated Cannabis sativa, MAJB-1, were extended onto 120-μm CG-71 and was packed into Column 1 of a 4-Column CG-71 system. Elution was done in recycle mode with 65% methanol and the column junctions were monitored by HPLC (Isco). When it was observed that Δ9-THC was just beginning to exit from Column 3, this column was isolated, the mobile phase was changed from 65% methanol to 75% methanol and a gradient was done from 75% methanol to 100% methanol in 100 minutes at 20 ml/minute. A fraction collector was set up to collect a fraction every 5 minutes so that each fraction would be 100 ml. Essentially all of the cannabinoids were eluted from Column 1; Column 2 contained some of the slow-moving Δ9-THCA as well as residual Δ9-THC and a small peak in the CBN retention time region. Column 3 contained the main quantity of Δ9-THC, and Column 4 contained only pure Δ9-THC, which had passed the junction between Column 3 and Column 4.

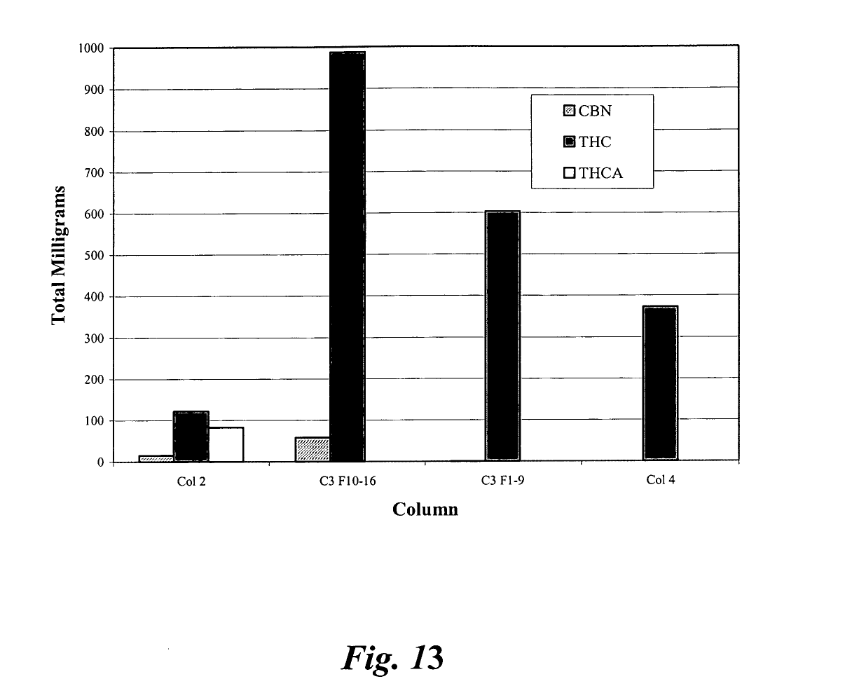

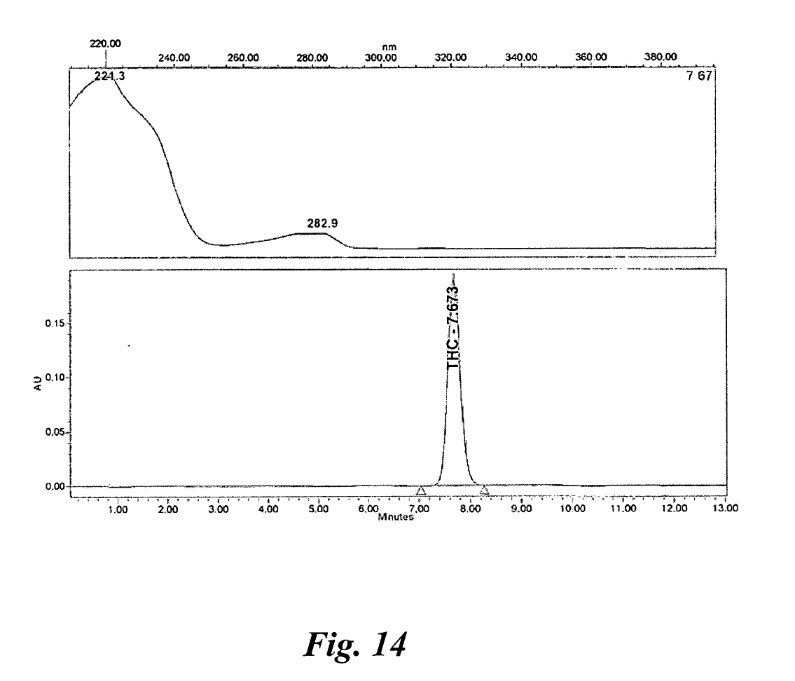

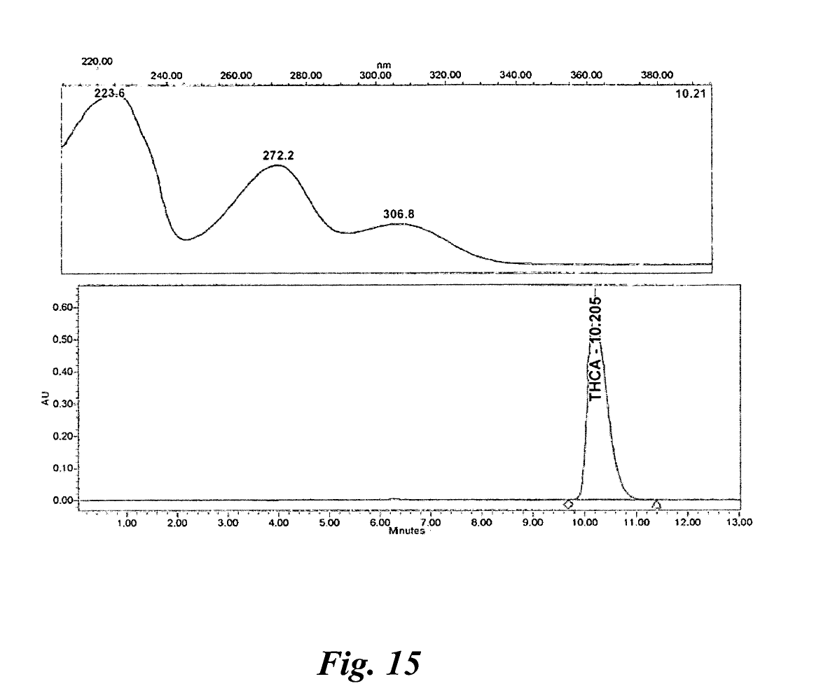

The results of the segmentation chromatographic separation of the SFS-CXP fractions of heat-treated Cannabis sativa, MAJB-1, are shown in FIG. 13. An HPLC chromatographic scan of Δ9-THC is shown as FIG. 14. The segmentation chromatographic system was also utilized to purify Δ9-THCA from the SFS-CXP fractions of untreated Cannabis sativa, MAJB-3. An HPLC chromatographic scan of Δ9-THCA is shown as FIG. 15.

A C18 (40 μm) segmentation chromatography 4-column system with a water:acetonitrile:acetic acid mobile phase was also developed and utilized for the separation of Δ9-THC from CBN, and for the polishing of high purity side-fractions. In this system, CBN was found to lead the Δ9-THC, which was opposite to the elution order found in the CG-71/methanol:water:acetic acid system.

While this invention has been particularly shown and described with references to specific embodiments, it will be understood by those skilled in the art that various changes in form and details may be made therein without departing from the spirit and scope of the invention as defined by the appended claims.

What is claimed is:

1. A prodrug composition isolated from marijuana species such as Cannabis sativa for treating pain and cachexia.

2. A composition of claim 1 consisting of Δ9-THCA that can be converted into Δ9-THC on heating to 100° C. or more.

3. A method for extracting and purifying natural cannabinoids such as Δ9-THCA and Δ9-THC from marijuana, which method consists of (a) extracting the biomass with a supercritical, near-critical or critical fluid with or without cosolvents and (b) purifying the cannabinoid-rich extracts by chromatography.

4. A method of claim 3 in which marijuana biomass is first heating to more than 100° C.

5. A method of claims 3 and 4 in which natural cannabinoids such as Δ9-THCA and Δ9-THC extracted from marijuana are (a) deposited onto the head of chromatography column; and (b) purifying the cannabinoid-rich extracts by chromatography.

6. A method of claim 5 in which the cannabinoids deposited onto the head of a chromatography column are eluted with a mobile phase consisting of supercritical, near-critical or critical fluid with or without cosolvents.

7. A method of claim 5 in which the cannabinoids deposited onto the head of a chromatography column are eluted with a mobile phase consisting of supercritical, near-critical or critical fluid with or without cosolvents and then purified with chromatography utilizing organic solvents.

8. A method of claim 5 in which the cannabinoids deposited onto the head of a chromatography column are eluted with and then purified with chromatography utilizing organic solvents.

9. A method for purifying cannabinoids involving the use of segmentation chromatography in which the target compounds are moved to column n leaving the faster-moving compounds on columns n+1 and the slower-moving compounds on columns n−1; column n is then eluted to yield purified fractions of the target compounds.

10. A method of claim 9 in which the mobile phase is recycled to move the target compounds from the n to the n+1 column.

11. Equipment of claim 3 for the supercritical, near-critical or critical fluid with or without cosolvents extraction of cannabinoids from marijuana in which the extracted cannabinoids are removed by flushing the pressure reduction device with high-pressure cosolvent.

12. Equipment of claim 4 for the supercritical, near-critical or critical fluid with or without cosolvents extraction and chromatographic purification of cannabinoids from marijuana in which the extracted cannabinoids are removed by flushing the pressure reduction device with high-pressure cosolvent.

13. Equipment of claim 9 for the chromatographic purification consisting of several individual columns designed to isolate different components of the deposited cannabinoids.

14. Equipment of claim 9 for the chromatographic purification consisting of a single column consisting of several individual segments designed to isolate different components of the deposited cannabinoids.

15. Equipment of claims 13 and 14 in which the mobile phase is recycled.
